(19) United States
(12) Patent Application Publication
(10) Pub. No.: US 2023/0165811 A1
(43) Pub. Date: Jun. 1, 2023

(54) METHOD FOR TREATING SYMPTOMS OF VIRAL INFECTIONS

(71) Applicant: Irving W. Wainer, Washington D.C., DC (US)

(72) Inventor: Irving W. Wainer, Washington D.C, DC (US)

(21) Appl. No.: 17/922,647

(22) PCT Filed: May 4, 2021
(86) PCT No.: PCT/US21/30577

§ 371 Date: Nov. 1, 2022

Related U.S. Application Data
(60) Provisional application No. 63/020,365, filed on May 5, 2020.

(51) Int. Cl. A61K 31/137; A61K 45/06

(52) U.S. Cl. CPC A61K 31/137; A61K 45/06

(57) ABSTRACT

A method for treating the symptoms of viral infections with fenoterol and/or fenoterol analogues.

This application is the U.S. National Stage filing of International Patent Application Number PCT/US2021/030577, filed on May 4, 2021, which claims the benefits of U.S. Provisional Patent Application Ser. No. 63/020,365 filed May 5, 2020, the contents of which are incorporated herein by reference.

### FIELD OF THE INVENTION

The present invention relates to a method for treating symptoms associated with viral infections. More specifically, the present invention relates to a method for treating symptoms such as systemic inflammation, hyperinflammation and cytokine storm syndrome associated with viral infections such as corona type viruses including severe acute respiratory syndrome (SARS) type viruses, middle east respiratory syndrome viruses (MERS) and SARS-CoV-2. The method comprises the administration of fenoterol and/or fenoterol analogues alone or combined with one or more therapeutic agents to a patient with a viral infection.

### BACKGROUND OF THE INVENTION

Corona type viruses are generally RNA type viruses that affect the respiratory system of mammals such as humans. The infections may be mild to severe. The infections may produce systemic inflammation, hyperinflammation and cytokine storm syndrome as well as renal and heart failure. In cases of severe infections, the patients may need to be place on mechanical ventilation.

There are currently no known vaccines or drugs that effectively prevent or treat corona type virus infections. Most treatments target the symptoms associated with viral infection. There is a need to improve upon the current treatment of patients with viral infections especially patients with SARS-CoV-2 infections.

### SUMMARY OF THE INVENTION

The present invention provides an improved and an alternative treatment for patients with viral infections by attenuating the patient's immune response and preventing hyperinflammatory and cytokine storm syndrome (CSS) thereby reducing further complications such as sepsis, acute respiratory distress, renal failure, and or cardiovascular issues. The present invention also relates to a method for slowing the clinical decline of patients with viral infections and thereby preventing, reducing or eliminating the need for mechanical ventilation.

The present invention comprise the step of administering a therapeutic amount of fenoterol and/or fenoterol analogues alone or combined with one or more therapeutic agents to a patient infected with a corona virus such as SARS-CoV-2 and MERS viruses.

In certain embodiments, the patient to be treated with the method of the present invention is infected with a corona virus and exhibits elevated inflammatory markers including but not limited to increased plasma levels of inflammatory cytokines, such as interleukin cytokines IL-6, IL-8, tumor necrosis factor-1a (TNF-1α), C-reactive protein (CRP), ferritin or a combination thereof. In some embodiments, the patient is infected with a corona virus and has lost a sense of smell and/or taste. The patient should be tested to confirm a corona viral infection prior to initiation of the treatment in accordance with the present invention.

The present invention further includes co-administering the therapeutic amount of fenoterol and/or fenoterol analogues in combination with a second therapeutic agent such as a high-mobility group box 1 (HMGB1) protein inhibitor, a Janus kinase (JAK) inhibitor, and immunosuppressant, an N-methyl-D-aspartate (NMDA) receptor inhibitor or combinations thereof.

### BRIEF DESCRIPTION OF THE DRAWINGS

FIG. 1 is a graph showing the attenuation of LPS-dependent TNF-α production in a murine RAW264.7 macrophage model of inflammatory response by (R.R′) 4′-methoxy-1-napthylfenoterol.

### DETAILED DESCRIPTION OF THE INVENTION

Before the present invention is further described, it is to be understood that this invention is not limited to the particular embodiments described herein. It is also to be understood that the terminology used herein is for the purpose of describing particular embodiments only and is not intended to be limiting.

It should be noted that as used herein, the singular forms “a,” “an,” and “the” include plural referents unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The terms “treating” and “treat” may be used interchangeably and include ameliorating, mitigating, and reducing the instances of one or more viral infection symptoms including but not limited to systemic inflammation, hyperinflammation, cytokine storm syndrome, sepsis, mechanical intubation-induced acute respiratory distress syndrome, renal failure, heart failure, and combinations thereof.

The terms “preventing” and “prevent” may be used interchangeably and include stopping one or more of the viral infection symptoms from presenting or progressing.

The terms “administering” and “administer” are used interchangeably and include any mode of administration, such as oral, subcutaneous, sublingual, transmucosal, parenteral, intravenous, intra-arterial, buccal, sublingual, topical, vaginal, rectal, ophthalmic, otic, nasal, inhaled, and transdermal. “Administering” can also include prescribing or filling a prescription for a dosage form comprising the therapeutic amount of fenoterol and/or fenoterol analogues and at least one pharmaceutically acceptable excipient. “Administering” can also include providing directions to carry out the method of the invention involving the therapeutic amount of fenoterol and/or fenoterol analogues or a dosage form comprising the therapeutic amount of fenoterol and/or fenoterol analogues and at least one pharmaceutically acceptable excipient.

As used herein, and unless otherwise defined, the terms “co-administration, “co-administered,” and “co-administer” refers to a subject or patient receiving (i) one or more fenoterol and/or non-fenoterol analogues and (ii) one or more drugs or therapeutic agents that is not a fenoterol or a fenoterol analogue during the course of the viral infection therapy. The one or more non-fenoterol and/or non-fenoterol analogues drugs or therapeutic agents may be administered concurrently or sequentially with the fenoterol and/or fenoterol analogues or dosage form of the present invention. The concurrent administration as used herein means the non-fenoterol or non-fenoterol analogue drug or therapeutic agent is administered within 1-4 hours before or after administration of the fenoterol and/or fenoterol analogues, preferably within 1 or 2 hours before or after administration of the fenoterol and/or fenoterol analogues and more preferably within 30 minutes or less before or after administration of the fenoterol and/or fenoterol analogues. The sequential administration as used herein means administration of the non-fenoterol and/or fenoterol analogue drug or therapeutic agent at any time before or after the administration of the fenoterol and/or fenoterol analogues and may include administration of the non-fenoterol and/or fenoterol analogue drug or therapeutic agent such as 4, 6, 8, 12, 14, 16, 18, 20, 22, 24 hours or longer before or after the administration of the fenoterol and/or fenoterol analogues.

As used herein, and unless otherwise defined, the term “subject” refers to a mammal such as a human, monkey, cow, horse, sheep, pig, chicken, turkey, quail, cat, dog, mouse, rat, rabbit, or guinea pig, preferably humans and includes healthy mammals and mammals affected with a disease that may be treated with the fenoterol and/or fenoterol analogues. A subject that is infected with a virus such as a corona virus that may be treated with the fenoterol and/or fenoterol analogues is sometimes referred to as “patient”.

As used herein the term fenoterol refers to a compound with the following structure:

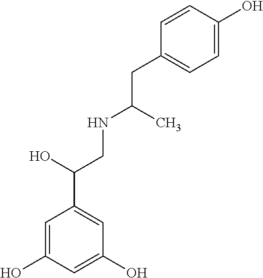 [SMILES: CC(Cc1ccc(O)cc1)NCC(O)c1cc(O)cc(O)c1]

As used herein the term fenoterol analogues refers to compounds with the following structure:

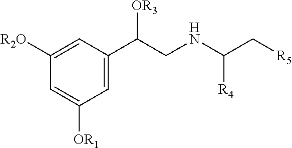

wherein R₁-R₃ independently are hydrogen, acyl, alkoxy carbonyl, amino carbonyl or a combination thereof;

R₄ is hydrogen or lower alkyl;

R₅ is lower alkyl,

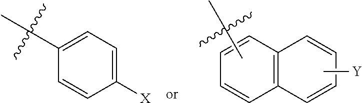

wherein X and Y independently are selected from hydrogen, —OR₆ and —NR₇R₈;

R₆ is hydrogen, lower alkyl or acyl; and

R₇ and R₈ independently are hydrogen, lower alkyl, alkoxy carbonyl, acyl or amino carbonyl.

In one embodiment, R₅ is a 1- or 2-naphthyl derivative optionally having 1, 2 or 3 substituents. Examples of such R₅ groups are represented by the formula

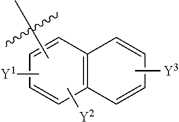

wherein V, Y² and Y³ independently are hydrogen, halogen, sulphur-containing moiety including SH, sulfoxides, sulphones, sulphanamides and related alkyl and aromatic substituted moieties, —OR₆ and —NR₇R₈;

R₆ is independently for each occurrence selected from hydrogen, lower alkyl and acyl. R₇ and R₈ independently are hydrogen, lower alkyl, alkoxy carbonyl, acyl or amino carbonyl (carbamoyl). In particular compounds at least one of Y¹, Y² and Y³ is —OCH₃, —OH or NH₂.

Particular R₅ groups include those represented by the formulas

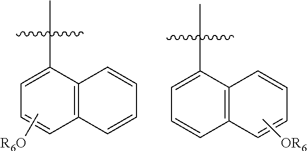

wherein R₆ is hydrogen, lower alkyl, such as methyl, ethyl, propyl or isopropyl or acyl, such as acetyl or NH₂.

Exemplary R₅ groups include

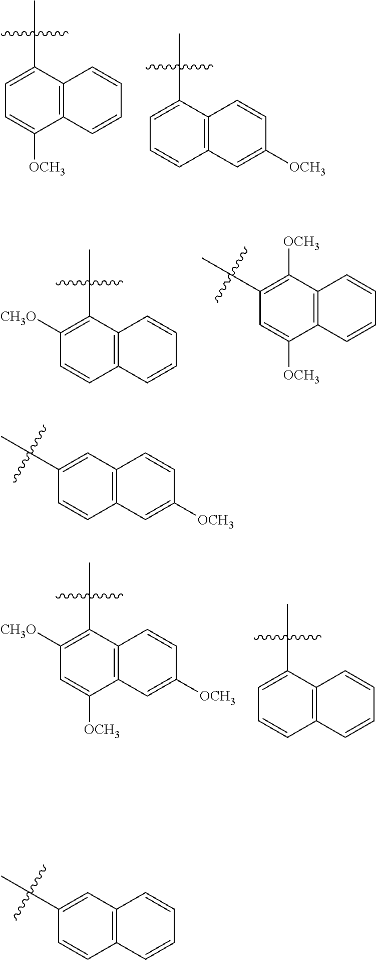

In one example, R₄ is lower alkyl and R₅ is

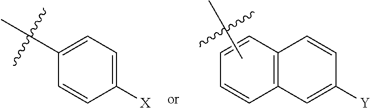

wherein X and Y independently are selected from hydrogen, lower alkyl—OR₆ and —NR₇R₈; R₆ is hydrogen, lower alkyl; and R₇ and R₈ independently are hydrogen or lower alkyl.

In a further example, R₄ is selected from ethyl, n-propyl, and isopropyl and R₅ has the formula

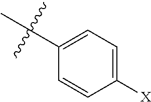

wherein X is hydrogen, —OR₆ or —NR₇R₈. For example, R₆ may be hydrogen, methyl or R₇ and R₅ are hydrogen.

In an additional example, R₅ has the formula

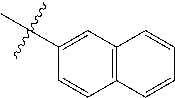 [SMILES: *c1ccc2ccccc2c1]

In further embodiments, R₄ is selected from methyl, ethyl, n-propyl and isopropyl and R₅ represents

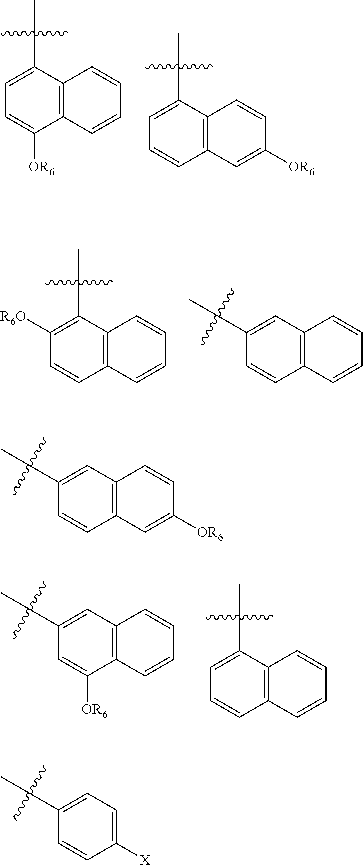

In some embodiments, R₁-R₃ independently are hydrogen; R₄ is a lower alkyl (such as, CH₃ or CH₂CH₃); R₅ is lower alkyl, or

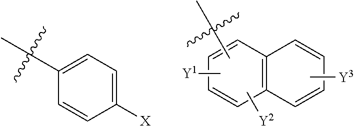

wherein X, Y¹, Y² and Y³ independently are hydrogen, —OR₆ and —NR₇R₈; R₆ is independently hydrogen, lower alkyl, acyl, alkoxy carbonyl or amino carbonyl; R₇ and R₈ independently are hydrogen, lower alkyl, alkoxy carbonyl, acyl or amino carbonyl and wherein the compound is optically active.

In some embodiments, R₁-R₃ independently are hydrogen; R₄ is a methyl or an ethyl; R₅ is

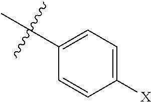

wherein X is —OH or —OCH₃.

In some embodiments, R₁-R₃ independently are hydrogen; R₄ is a methyl or an ethyl; R₅ is

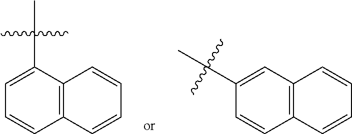 [SMILES: *c1cccc2ccccc12 ; *c1ccc2ccccc2c1]

In some embodiments, the fenoterol analogues used in the present invention have the following structures:

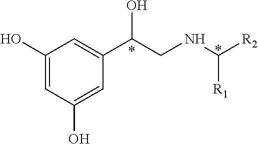

| | | | Abbre- |
|---|---|---|---|
| | | | via- |
| Compound | R1 | R2 | tion |
| 4′-methoxy-1- naphthylfenoterol | CH₃ | 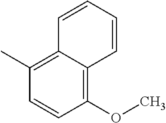 | MNF |
| 4′-hydroxy-1- naphthylfenoterol | CH₃ | 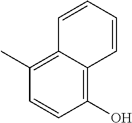 | HNF |
| 4′-amino-1- naphthylfenoterol | CH₃ | 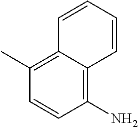 | ANF |
| 4′-methoxy-1- naphthylethyl- fenoterol | CH₂CH₃ | 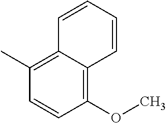 | MNEF |
| 4′-hydroxy-1- naphthylethyl- fenoterol | CH₂CH₃ | 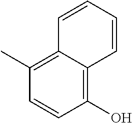 | HNEF |
| 4′-amino-1- naphthylethyl- fenoterol | CH₂CH₃ | 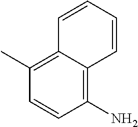 | ANEF |
| 1- napthylfenoterol | CH₃ | 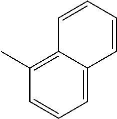 | 1NF |
| 1-napthylethyl- fenoterol | CH₂CH₃ | 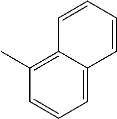 | 1NEF |
| 2- napthylfenoterol | CH₃ | 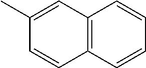 | 2NF |
| 2-napthylethyl- fenoterol | CH₂CH₃ | 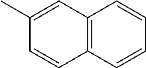 | 2NEF |

In some embodiments, the fenoterol analogues include specific isomers of the above compounds such as:

(R,R′)-MNF;

(R,S′)-MNF;

(R,R′)-HNF;

(R,S′)-HNF;

(R,R′)-ANF;

(R,S′)-ANF;

(R,R′)-MNEF;

(R,S′)-MNEF;

(R,R′)-HNEF;

(R,S′)-HNEF;

(R,R′)-ANEF;

(R,S′)-ANEF;

(R,R′)-1NF;

(R,S′)-1NF;

(R,R′)-1NEF;

(R,S′)-1NEF;

(R,R′)-2NF;

(R,S′)-2NF;

(R,R′)-2NEF; and

(R,S′)-2NEF.

The isomers may be administered in a pure or substantially pure form. As used herein the term “substantially pure” means the fenoterol and fenoterol analogues comprise at least 85% of the specific isomer, preferably at least 90% of the specific isomer and more preferably at least 95% of the specific isomer. As used herein the term pure means the fenoterol and fenoterol analogues comprise at least 97% of the specific isomer, preferably at least 98% of the specific isomer and more preferably at least 99% of the specific isomer.

Process for preparing fenoterol and fenoterol analogues are provided in U.S. Pat. Nos. 10,485,771 and 10,562,843, which are incorporated herein by reference.

The fenoterol and fenoterol analogues may be administered as a free base or as pharmaceutically acceptable salt. The salts are generally pharmaceutically acceptable salts that are non-toxic. Salts may be of any type (both organic and inorganic), such as fumarates, tartrates, tosylates, hydrobromides, hydrochlorides, sulfates and phosphates.

Additional examples of salt-forming groups include, but are not limited to, a carboxyl group, a phosphonic acid group or a boronic acid group, that can form salts with suitable bases. These salts can include, for example, nontoxic metal cations, which are derived from metals of groups IA, IB, IIA and IIB of the periodic table of the elements. In one embodiment, alkali metal cations such as lithium, sodium or potassium ions, or alkaline earth metal cations such as magnesium or calcium ions can be used. The salt can also be a zinc or an ammonium cation. The salt can also be formed with suitable organic amines, such as unsubstituted or hydroxyl-substituted mono-, di- or tri-alkylamines, in particular mono-, di- or tri-alkylamines, or with quaternary ammonium compounds, for example with N-methyl-N-ethylamine, diethylamine, triethylamine, mono-, bis- or tris-(2-hydroxy-lower alkyl)amines, such as mono-, bis- or tris-(2-hydroxyethyl)amine, 2-hydroxy-tert-butylamine or tris(hydroxymethyl)methylamine, N,N-di-lower alkyl-N-(hydroxy-lower alkyl)amines, such as N,N-dimethyl-N-(2-hydroxyethyl)amine or tri-(2-hydroxyethyl)amine, or N-methyl-D-glucamine, or quaternary ammonium compounds such as tetrabutylammonium salts.

Exemplary compounds disclosed herein possess at least one basic group that can form acid-base salts with inorganic acids. Examples of basic groups include, but are not limited to, an amino group or imino group. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with α-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid. In a currently preferred embodiment, fenoterol is provided as a hydrobromide salt and exemplary fenoterol analogues are provided as their fumarate or tartrate salts.

Additional counter ions for forming pharmaceutically acceptable salts of fenoterol and fenoterol analogues may be found in the Handbook of Pharmaceutical Salts, Properties, Selection, and Use, edited by Stahl et al. 2002, p. 330 and Berge et al., “Pharmaceutical Salts,” Journal of Pharmaceutical Sciences, Vol. 66, No., 1977, pp. 1-19.

The present invention comprise the step of administering a therapeutic amount of fenoterol and/or fenoterol analogues alone or combined with one or more therapeutic agents to a patient infected with a corona virus such as SARS-CoV-2 and MERS viruses. The fenoterol and/or fenoterol analogues is preferably selected from the group consisting of MNF, HNF, ANF, MNEF, HNEF, ANEF, 1NF, 1NEF, 2NF, 2NEF, pharmaceutically acceptable salts thereof, pure or substantially pure isomers thereof or combinations of the foregoing.

Fenoterol and the fenoterol analogues are known to exhibit activity at two G protein-coupled receptors, GPR55 inhibitor and the β2-adrenergic receptor (activator). Both receptors are associated with inflammatory immune responses and increased and activated by viral infections. Fenoterol and fenoterol analogues such as MNF HNF, ANF, MNEF, HNEF, ANEF, 1NF, 1NEF, 2NF, 2NEF competitively inhibit GPR55 and activates the β2-adrenergic receptor and both activities are associated with a decreased production of inflammatory cytokines. Therefore it is believed that the administration of fenoterol and fenoterol analogues to a patient with a viral infection such as a corona virus will reduce the patient's inflammatory response to the infection and hopefully prevent or attenuate the more severe sides effects of the viral infection such as cytokine storm syndrome, sepsis, mechanical intubation-induced acute respiratory distress syndrome, renal failure and cardiac failure.

The administration of the fenoterol and/or fenoterol analogues may be as single or multiple administrations of dosage forms comprising the fenoterol and/or fenoterol analogues and one or more pharmaceutically acceptable excipients. Generally, multiple administrations are administered once, twice, three or four times a day and the administrations or treatment will last for as long as the patient tests positive for the viral infection and may occur for at least one, two, three, four, five, six, seven, eight weeks or longer.

Therapeutically effective doses of a disclosed fenoterol and/or fenoterol analogues can be determined by one of skill in the art. An example of a dosage range is from about 0.001 to about 10 mg/kg body weight of the patent when the fenoterol and/or fenoterol analogue is administered orally in single or divided doses.

The administration of the therapeutic amount of fenoterol and/or fenoterol analogues may occur orally including buccally, sublingually, parenterally including intravenously, subcutaneously, intra-arterially, transmucosally including vaginally, rectally, optically, otically, nasally, via inhalation, transdermally/topically or combination thereof.

In certain embodiments, the administration of the therapeutic amount of fenoterol and/or fenoterol analogues is preferably orally as immediate, delayed or sustained formulations via a solid dosage form such as a tablet, capsule, powder or a liquid dosage form such as a syrup, solution or suspension. In particular examples, a dosage range of the fenoterol and/or fenoterol analogues is from about 0.005 to about 5 mg/kg body weight administered orally in single or divided doses (assuming an average body weight of approximately 70 kg; values adjusted accordingly for persons weighing more or less than average). For oral administration, the oral dosage forms comprise about 1.0 mg to about 500 mg, about 1.0 mg to about 250 mg or about 1.0 mg to about 100 mg of the fenoterol and/or fenoterol analogues. In one embodiment of the present invention the patient is orally administered about 1 mg to about 50 mg of the fenoterol and/or fenoterol analogues in the form of a tablet, capsule, powder, syrup, solution or suspension one to six times a day, preferably two times, three times or four times a day, i.e. every 12 hours, every 8 hours, or every 6 hours.

In an alternative embodiment, the administration of the therapeutic amount of fenoterol and/or fenoterol analogues is preferably parenterally such as intravenously, subcutaneously or intra-arterially via sterile solution or suspension. A suitable dose for parental administration is about 1 milligram per kilogram (mg/kg) to about 100 mg/kg, such as a dose of about 10 mg/kg to about 80 mg/kg, such including about 1 mg/kg, about 2 mg/kg, about 5 mg/kg, about 20 mg/kg, about 30 mg/kg, about 40 mg/kg, about 50 mg/kg, about 80 mg/kg or about 100 mg/kg administered parenterally. However, other higher or lower dosages also could be used, such as from about 0.001 mg/kg to about 1 g/kg, such as about 0.1 to about 500 mg/kg, including about 0.5 mg/kg to about 200 mg/kg.

The fenoterol and/or fenoterol analogues will be administered in accordance with the present invention in an amount that will attenuate the pro-inflammatory response associated with the corona virus infection, especially SARAS-CoV-2 infections and increase the expression of anti-inflammatory cytokines thereby allowing the patient's immune system to clear the viral infection without progressing to a hyperinflammatory immune state, which may include cytokone storm syndrome and/or mechanical intubation-induced acute respiratory distress syndrome (ARDS). This attenuation will occur by allowing the fenoterol and/or fenoterol analogues to inhibit GPR55 receptor and activate the β2-adrenergic receptors.

The present invention further include co-administering the therapeutic amount of fenoterol and/or fenoterol analogues in combination with one or more second therapeutic agents such as a high-mobility group box 1 (HMGB1) protein inhibitor, a Janus kinase (JAK) inhibitor, and immunosuppressant, an N-methyl-D-aspartate (NMDA) receptor inhibitor or combinations thereof. The co-administration may be concurrently or sequentially.

Examples of HMGB1 protein inhibitors include heparin, ascorbic acid.

Examples of JAK inhibitors include ruxolitinib, tofacitinib, oclacitinib, baricitinib, peficitinib, fedratinib, upadacitinib, filgotinib, cerulatinib, gandotinib, lestaurtinib, momelotinib, pacritinib and abtocotinib.

Examples of immunosupressant include anakinra (IL-1), tocilizumab (IL-6), prednisone, cyclosporine, methotrexate, apremilast, azathioprine, mycophenolate mofetil, tacrolimus, sirolimus, everolimus, infliximab, adalimumab, etanercept, and ustekinumab.

Examples of NMDA receptor inhibitors include pethidine, levorphanol, methadone, dextropropoxyphene, tramadol, ketobemidone, ketamine, ketamine analogues, dextromethorphan, phencyclidine, and methoxetamine. Ketamine analouges such as (2R,6R)-hydroxynorketamine are described in U.S. Pat. Nos. 9,650,352 and 9,867,830 which are incorporated herein by reference.

Dosage Forms

The dosage forms comprising the fenoterol and/or fenoterol analogues that are administered to the patient in accordance with the present invention comprise (i) an effective amount of at least one fenoterol and/or fenoterol analogue, preferably MNF, HNF, ANF, MNEF, HNEF, ANEF, 1NF, 1NEF, 2NF, 2NEF, substantially pure or pure isomers thereof, pharmaceutically acceptable salts thereof or a combination the foregoin and (ii) at least one pharmaceutically acceptable carrier or excipient; The pharmaceutically acceptable carrier(s) or excipient(s) are known in the art and their selection will depend upon the route of administration.

Oral Dosage Forms

The oral dosage forms that may be used in the present invention include both solid and liquid dosage forms. Solid dosage forms include but are not limited to tablets, capsules, pellets, granules, powders. The liquid dosage forms include syrups, solutions and suspensions. The oral dosages forms may be swallowed or applied to the oral cavity, i.e., sublingually, lingually or buccally. The oral dosage forms may be formulated to be immediate release, delayed release, controlled release, or a combination thereof.

In embodiments where the oral dosage form is a solid dosage form, the at least one fenoterol and/or fenoterol analogues may be combined with pharmaceutically acceptable excipients such as fillers, diluents, binders, stabilizing agents, lubricants, disintegrants or mixtures thereof. These pharmaceutically acceptable excipients are well known in the art and are described in Remington, the Science and Practice of Pharmacy, 21″ Ed. (2006), pp. 1058-1092, published by Lippincott Williams & Wilkins; United States Pharmacopeia 27 (2004), pp. 2809-2812; and Handbook of Pharmaceutical Excipients, 5^{th} Ed. (2006), published by the Pharmaceutical Press, both incorporated by reference. The solid oral dosage forms are made by methods commonly known in the art such as direct compression, wet or dry granulation, and extrusion spheronization.

Examples of acceptable fillers, sometimes referred to as diluents, include sugars such as lactose, dextrose, sucrose, maltose, or microcrystalline cellulose, clays, and mixtures thereof.

Examples of binders that are useful in the present invention include pharmaceutically acceptable substances with cohesive properties. Some examples include celluloses such as hydroxypropyl methycellulose, hydroxypropyl cellulose and carboxymethycellulose sodium, polyvinylpyrrolidone, sugars, starches, and mixtures thereof.

Examples of stabilizing agents that are useful in the present invention include organic acids and alkaline metal salts of organic acids, such as succinic acid, fumaric acid, citric acid, sodium citrate, and mixtures thereof; antioxidants such as butylated hydroxyanisole, butylated hydroxytoluene, hypophosphorous acid, monothioglycerol, potassium metabisulfate, propyl gallate, sodium bisulfite, sodium formaldehyde sulfoxylate, sodium metabisulfate, sodium sulfate, sodium thiosulfate, sodium dioxide, tocopherol and mixtures thereof and chelating agents such as citric acid as well as its salt forms, polyphosphates (e.g., sodium tripolyphosphate, hexametaphosphoric acid, sodium acid pyrophosphate, sodium pyrophosphate, tetra sodium pyrophosphate, sodium hexametaphosphate, sodium metaphosphate); aminocarboxylic acids (e.g., ethylenediaminetetraacetic acid (EDTA) or combinations thereof.

Examples of lubricants, glidants and/or antiadherents that may be used in the present invention include talc, magnesium stearate, calcium stearate, stearic acid, hydrogenated vegetable oils, polyethylene glycols, silicon dioxide, and mixtures thereof.

Examples of disintegrating agents that can be used in the present invention include corn starch, croscarmelose sodium, crospovidone (polyplasdone XL-10), sodium starch glycolate (EXPLOTAB® or PRIMOJEL), or any combination of the foregoing.

In embodiments wherein the oral dosage form is a liquid dosage form such as a syrup, solution or suspension, the liquid dosage form typically contain pharmaceutically acceptable excipients such as a liquid carrier, i.e., water and/or alcohol, pharmaceutically acceptable solvents, flavoring agents, stabilizing agents, coloring agents, viscosity increasing agents or mixtures thereof. The pharmaceutically acceptable excipients employed in the syrups, solutions or suspensions of the present invention are described in Remington, the Science and Practice of Pharmacy, 21″ Ed. (2006), pp. 745-775, published by Lippincott Williams & Wilkins; United States Pharmacopeia 27 (2004), pp. 2809-2812; and Handbook of Pharmaceutical Excipients, 5th Ed. (2006), published by the Pharmaceutical Press, incorporated by reference and further described below.

Examples of liquid carriers that may be used are alcohols such as ethanol, alcohol-water mixtures, and low molecular weight polymers such as polyethylene glycol.

Examples of flavoring agents that may be used in the present invention include peppermint, spearmint, wintergreen, cinnamon, coconut, coffee, chocolate, vanilla, menthol, licorice, anise, apricot, caramel, pineapple, strawberry, raspberry, grape, cherry, mixed berry, tropical fruits, mint, and mixtures thereof.

Examples of coloring agents that may be employed in the present invention include FD&C-type dyes and lakes, fruit and vegetable extracts, titanium dioxide, and mixtures thereof.

Examples of viscosity increasing agents have been described previously, and a few representative examples that may be included in the liquid dosage forms include methylcellulose, xanthan gum, carboxymethyl cellulose, hydroxypropyl cellulose, carbomer, acacia, agar, alginate, carrageenan, gum tragacanth, collagen, carboxypolymethylene, glyceryl monostearate, monostearate, polyvinylpyrrolidone, polyacrylamide, and mixtures thereof.

Parenteral Dosage Forms

The parenteral dosage forms that may be used in the present invention include dosage forms for administration of the fenoterol and/or fenoterol analogues by intravenous, subcutaneous, intradermal, intramuscular, intraarticular or intrathecal injections. The parenteral formulation may comprise fenoterol and/or fenoterol analougue and water for injection and optionally one or more additional pharmaceutically acceptable excipients such a preservative, a pH adjusting agent, an osmolarity adjusting agent and combinations thereof. The parenteral formulation may be packaged in vials or may be in the form of a dry powder that is packaged in a vial and that may be reconstituted with water.

Inhalation Dosage Forms

The inhalation dosage forms that may be used in the present invention will deliver the fenoterol and/or fenoterol analogues to the respiratory system of the patient, preferably the lungs and may be in the form of an inhalable powder, solution or suspension.

The inhalable powders, solutions and suspensions will comprise the fenoterol and/or fenoterol analogues for delivery to the respiratory system of the patient and may be prepared by any means commonly employed in the art, and employ excipients as described above for preparation of the solid and liquid oral dosage forms. The inhalation dosage forms may be delivered to a patient's respiratory system, using apparatuses commonly known in the art such as those described in U.S. Pat. Nos. 5,349,945; 5,394,868; 5,674,472; 5,766,573; 5,860,419; 6,641,800; 6,521,212; 9,339,507 and U.S. Patent Publication No. 2004/0265238 which are incorporated herein by reference.

Nasal Dosage Forms

The nasal dosage forms that may be used in the present invention may be in the form of a liquid, preferably a solution or suspension that can be sprayed onto or applied to the nasal passages via drops or swabs. In addition to the fenoterol and/or fenoterol analogues the nasal dosage forms may contain inert diluents and/or solvents commonly used in the art. Water is the preferred solvent, however, combinations of water with other physiologically acceptable solvents may also be employed. Other solvents, solubilizing agents and emulsifiers suitable for use in place of, or in addition to, water include but are not limited to saturated aliphatic mono- and polyvalent alcohols which contain 2-6 carbon atoms (including, but not limited to, ethanol, 1,2-propylene glycol, sorbitol, and glycerine), polyglycols such as polyethylene glycols, and surfactants/emulsifiers like the fatty acid esters of sorbitan, and mixtures thereof. Oils, in particular, cottonseed, peanut, or corn oils, may also be added to the dosage forms. The combination of the additional solvents in the aqueous solution should preferably not exceed about 15% (w/v) of the total dosage form.

The nasal dosage forms of the present invention may further comprise one or more preservatives and/or one or more stabilizers as previously described.

It is desirable that the nasal dosage forms of the present invention exhibit a pH of about 4.5 to about 7.4, and preferably have a pH of about 5.5 to 7.1, for physiological reasons. Accordingly, in additional embodiments of the present invention, the dosage forms of the invention may further comprise one or more buffering agents that are used to adjust and/or maintain the dosage form in the desired pH range. Examples of pH or buffering agents that are suitable for use in the dosage forms of the invention include, but are not limited to, citric acid, sodium citrate, sodium phosphate (dibasic, heptahydrate form), and boric acid or equivalent conventional buffers, and combinations thereof. The appropriate amounts of buffers and buffering agents, or combinations thereof, that are to be used in the dosage forms of the invention are described in the United States Pharmacopoeia, Remington: The Science and Practice of Pharmacy, and the like, the disclosures of which are incorporated herein by reference in their entireties.

The nasal dosage forms of the invention may also further comprise one or more taste-masking agents, one or more flavoring agents, one or more sweetening agents, and/or a combination of such agents.

In an embodiment of the invention, the nasal dosage forms may further comprise one or more water-soluble viscosity-increasing agents. Such agents are preferably used at the concentration of about 0.01% to about 5.0% (w/v), in order to typically produce a viscosity of the final solution between about 2 and about 300 centipoise. Viscosity-increasing agents that are suitable for use in accordance with the present invention include, but are not limited to, polyvinylpyrrolidones, cellulose derivatives including, but not limited to, hydroxyethyl cellulose, carboxymethyl cellulose or its salts, hypromellose, carrageenan, guar gum, alginates, carbomers, polyethylene glycols, polyvinyl alcohol, and xanthan gum.

Topical Dosage Forms

The topical dosage forms that may be used in the present invention include occluded forms, such as matrix and reservoir patches, and unoccluded forms, such as gels, creams, lotions, ointments, and serums, as wells as topical foams and mousses.

Matrix patches in accordance with the present invention comprise at least one fenoterol and/or fenoterol analogue homogeneously blended in a solid or semisolid polymer carrier together with other additives (e.g., permeation enhancers, plasticizers, viscosity reducing agent, and the like). The general structure and fabrication of matrix patches are well known in the art. In a preferred embodiment, the matrix patch comprises an occlusive backing that is impermeable to the at least one fenoterol and/or fenoterol analogue and defines the face or top surface of the patch and a solid or semisolid matrix layer comprised of a homogeneous blend of the at least one fenoterol and/or fenoterol analogue and one or more skin permeation enhancers.

The polymeric carrier may be adhesive or nonadhesive. When it is a pressure sensitive adhesive the basal surface of the matrix layer may be used to affix the patch to the skin. When it is not, other means such as an underlying adhesive layer, a peripheral adhesive layer, an adhesive overlay, or straps may be used to affix the patch to the skin.

Examples, without limitation, of specific polymers that may be used as the carrier are polyacrylates, polymethacrylates, natural and synthetic rubbers, silicone rubbers and elastomers, polyolefins, vinyl copolymers, urethanes, nylons, polyesters, polyethers, and the like.

The skin permeation enhancer(s) that are included in the matrix enhance the level of skin flux of fenoterol and/or fenoterol analogue. Examples of permeation enhancers that may be used in transdermal dosage forms of the present invention include, but are not limited to, fatty acids, fatty acid esters, fatty alcohols, fatty acid esters of lactic acid or glycolic acid, glycerol tri-, di- and monoesters, triacetin, short chain alcohols, amine oxides and mixtures thereof. Particular examples of permeation enhancers include oleyl alcohol, lauryl alcohol, isopropyl myristate, oleyl oleate, levulinic acid, ethanol, glycerol monooleate, methyl laurate, sorbitain monooleate, triacetin, aloe vera oil, benzothonium chloride, cetyl dimethylamine oxide, cetyl alcohol, cetyl lactate, cocamidopropyl betaine, cocoamine oxide diethanolamine, dimethyloctylamine oxide, 2-dodecoxyethyldimethylamine oxide, dimethyl-decylamine oxide, dimethylhexadecylamine oxide, dimethyl-tetradecylamine oxide, dimethyl isosorbide, dipropylene glycol, ethyl hexyl lactate, glycolic acid, 3-dodecoxy-2-hydroxypropyldi(3-hydroxypropyl)amine oxide, lactic acid, lauramine oxide, lauryl betaine, lauryl lactate, lauryl laurate, isopropyl palmitate, macrogol 15 hydroxystearate (Solutol HS 15), menthol, menthyl lactate, myristyl alcohol, myristal lactate, octyldodecanol, octyl salicylate, oleamine oxide, oleic acid, oleyl betaine, oleyldi(2-hydroxyethyl) amine oxide, PEG 1000, pentadecalactone, propylene glycol, salicylic acid, stearyl alcohol, stearyl lactate, 3,6,9-trioxaheptadecyldiethylamine oxide, di(2-hydroxyethyl)-tetradecylamine oxide, triethanolamine triacetate and combinations thereof. Other permeation enhancers useful with the present invention may be found in U.S. Patent Application Publication No. 2007/0269379, which is incorporated in its entirety herein by reference.

The patches of the invention may be manufactured by conventional techniques used in transdermal drug delivery device art. For instance, the at least one fenoterol and/or fenoterol analogue, carrier, and enhancer(s) may be mixed in the desired proportions to form a homogeneous mixture and cast or otherwise applied to a backing layer, by lamination to a release liner layer.

Reservoir patches in accordance with the present invention may comprise a gelled liquid solution or suspension containing at least one fenoterol and/or fenoterol analogue and an enhancer within a carrier or be in the form of a fibrous body impregnated with the drug in the carrier. In addition to the reservoir, the device includes means for maintaining the reservoir in diffusional communication with the skin. Such means include a carrier which is also an adhesive, a separate basal adhesive layer underlying the reservoir, a peripheral ring of adhesive that is interconnected to the reservoir, an adhesive overlay for the reservoir, and straps. Preferably the means is either an adhesive carrier or a separate underlying adhesive layer.

In addition to the reservoir and affixation means, the patches may further include a backing that overlies the reservoir and protects the reservoir and/or prevents back-diffusion of the at least one fenoterol and/or fenoterol analogue and enhancer from the reservoir, one or more structural layers to provide the device with appropriate mechanical properties, and/or a release liner layer that underlies the reservoir and which is removed prior to use and means for affixing the device to the skin.

The carrier is a fluid (e.g., liquid, gel, emulsion, suspension). It may be aqueous or nonaqueous. Examples of fluid carriers that may be used are alcohols such as ethanol, alcohol-water mixtures, and low molecular weight polymers such as polyethylene glycol. Ethanol is preferred and also provides permeation enhancement. In the case of ethanol, the carrier normally constitutes 20% to 70% by volume of the reservoir, more usually 40% to 60%, and preferably approximately 50%. Alternatively, the carrier may be a solid or semisolid matrix such as a pressure-sensitive adhesive.

The reservoir patches may contain a permeation enhancer as discussed above. The reservoir may also contain amounts of other materials such as gelling agents and anti-irritants. Glycerin is a preferred anti-irritant and may be present at 5% to 50%, preferably 20% to 30% by volume.

The reservoir patches may be manufactured by conventional techniques used in the transdermal drug delivery device art. For instance, at least one fenoterol and/or fenoterol analogue and carrier may be mixed in the desired proportions to form a homogeneous mixture and cast or otherwise applied to a backing layer, followed by lamination to a release liner layer. If a separate basal adhesive layer is desired, it may be cast onto the release liner layer prior to such lamination.

The patches will be typically designed to be worn for 0.5 to 14 days, more preferably 1 to 7 days, and most preferably 1-3 days. The area of the patch in diffusional contact with the skin may be between 1 and 150 cm², more preferably 5 and 100 cm², and most preferably 10 and 75 cm². The required dosing may be supplied by a single device or by a plurality of devices applied to the skin.

Further embodiments of the topical dosage forms that may be used in the present invention include topical gels, creams, lotions, ointments, serums, foams, and mousses (collectively “unoccluded topical dosage forms”).

In addition to the at least one fenoterol and/or fenoterol analogue, the unoccluded topical dosage forms may contain a penetration enhancer as discussed above. Depending upon the specific topical dosage form, i.e., serum, cream or foam, the topical dosage form of the present invention may also include further additives such as solvents, film forming/polymeric agents, viscosity increasing agents, emulsifiers, antioxidants, preservatives, pH adjusting agents, propellants and combinations of the foregoing. The unoccluded topical dosage forms may be uniform compositions, emulsions, such as oil-in-water or water-in-oil emlusions, or liposomal compositions.

The unoccluded topical dosage forms of the present invention may include any suitable solvent. Preferably, the solvent may include water and/or one or more organic compounds, e.g., esters, alcohols, ketones, aldehydes, fatty acids, partially or fully esterified fatty acids, wherein the structures are cyclic, non-cyclic (e.g., alkyl), alicyclic (i.e., a bridged ring compound), or aromatic, as well as organic compounds having combinations of these functional groups. Specific examples of solvents that may be employed are water, methanol, ethanol, isopropyl alcohol, acetone, hexane, butyl alcohol, ethyl acetate, polyethylene glycol, propylene glycol, ethylene glycol, triethylene glycol, glycerin, 1,3-propane diol, 2-methyl-1,3-propane diol, glycerol ricinoleate, mineral oil, peanut oil, corn oil, cottonseed oil, sesame oil or a combination thereof. The solvent may be employed in any suitable amount. Typically, the solvent can be present in the unoccluded topical dosage forms in about 1.0 wt % to about 95.0 wt % based upon the total weight of the unoccluded topical dosage form, preferably about 3.0 wt % to about 85 wt % based upon the total weight of the unoccluded topical dosage form and most preferably about 5.0 wt % to about 75 wt % of the total weight of the unoccluded topical dosage form.

The unoccluded topical dosage forms of the present invention also may optionally include a film-forming/polymeric agent. The film-forming/polymeric agent may enhance the adherence of the composition to the patient's skin and improve the composition's resistance to washing off or rubbing off. Film-forming/polymeric agents are preferably soluble or miscible with the at least one cannabinoid, the at least one terpene or the combination of at least one cannabinoid and at least one terpene, solvent and/or penetration enhancer. The unoccluded topical dosage forms of the present invention typically comprises from about 0.001 wt % to about 25 wt %, preferably about 0.005 wt % to about 15 wt % and most preferably about 0.010 wt % to about 10 wt % based upon the total weight of the unoccluded topical dosage form of the film-forming/polymeric agents. Some examples of film-forming/polymeric agents that may be used in topical dosage forms of the present invention are polyalkenes, oleophilic copolymers of vinvylpyrrolidone, acrylic copolymers, polyethylene glycol derivative, polyolefins, polyurethanes and mixtures thereof. Additional examples of film-forming agent can be found in U.S. Pat. No. 10,214,013, which is incorporated herein by reference.

The unoccluded topical dosage forms of the present invention may also contain viscosity enhancing agents that thicken, gel or harden the unoccluded dosage form. An unoccluded topical dosage forms in accordance with the present invention, such as a topical gel, typically comprises from about 0.001 wt % to about 50 wt % of the viscosity enhancing agent, preferably about 0.005 wt % to about 40 wt % and most preferably about 0.01 wt % to about 25 wt % based upon the total weight of the unoccluded topical dosage form. Exemplary viscosity enhancing agents include organic materials such as natural or synthetic waxes, C12-C60 alcohols, C12-C60 acids, alpha-hydroxy fatty acids, polyhydroxy fatty acid esters, polyhydroxy fatty acid amides, and inorganic/organic materials such as metal ester complexes containing zinc, calcium, aluminum or magnesium, fumed silicas, and organoclays. Additional viscosity enhancing agents include polyol polyesters, glyceryl esters, polyglyceryl esters and polysiloxanes that are a solid or semi-solid at ambient temperature. Additional examples of viscosity enhancing agents can be found in U.S. Pat. No. 10,214,013, which is incorporated herein by reference.

If the unoccluded topical dosage form of the present invention is an aerosol, foam or mouse, the composition will require a propellant for dispensing the composition from the container. The propellant may be any type of propellant commonly used in the cosmetic/pharmaceutical industry such as nitrogen, carbon dioxide, dimethyl ether, hydrocarbons, i.e., methane, ethane, propane, butanes and pentanes, halogenated hydrocarbons, i.e., CH₂ClF, CClF₂CHClF, CF₃CHClF, CHF₂CClF₂, CHClFCHF₂, CF₃CH₂Cl, CClF2CH₃, CHF₂CHF₂, CF₃CH₂F (HFC 134a), CHF₂CH₃ (HFC 152a), CF₃CHFCF₃ (HFC 227), CF₃CF₃ and CF₃CF₂CF₃. Some of the more commonly used hydrocarbon propellants are A-46 (15.2% propane/84.8% isobutene); and NP-46 (25.9% propane/74.1% n-butane), NIP-46 (21.9% propane/31.3% isobutene/46.8% n-butane). The amount of propellant will depend on the type of container for the composition of the present invention, the amount of the composition in the container, the amount of composition to be dispensed per actuation and the form in which the composition will be dispensed, i.e., mist or foam. The optimization of the propellant and container are within the ability of the skilled artisan and examples can be found in Wai-Chiu So et al., U.S. Pat. No. 6,946,120 and Remington, Science and Practice of Pharmacy, 21″ ed., pp. 1000-1017 which are incorporated in their entireties herein by reference.

The aerosols, foams and mousses of the present invention will include a solvent, preferably water and/or a lower alcohol, i.e., C1-C6 alcohols such as methanol, ethanol, isopropanol or mixtures thereof. The aerosols, foams or mousses may also comprise a co-solvent selected from one or more of the group consisting of aromatic and polyhydric alcohols such as 1,3-butylene glycol, propylene glycol, polyethylene glycol 400, hexylene glycol and dipropylene glycol or glycerol. When the co-solvent is present, it may be present in amounts of approximately 10% by weight or less, preferably approximately 5% by weight or less based upon the total weight of the composition.

Additional examples of the excipients that may be used in unoccluded dosage forms and methods for making various unoccluded dosage forms that may be used in the methods of the present invention can be found in U.S. Pat. No. 10,214,013 which is incorporated herein by reference.

Transmucoal Dosage Forms

The transmucoal dosage forms that may be used in the present invention include gels creams, lotions, solutions, suspensions, suppositories that may be applied to mucosal surfaces such as the vaginal or rectal areas. The transmucosal gels, creams, solutions, or suspension may be prepared as described above for the unoccluded transdermal dosage forms. Some examples of the transmucosal gels, creams, solutions, or suspension may be prepared can be found in U.S. Pat. Nos. 8,658,678 and 8,946,276 which are incorporated herein by reference.

Examples of suppository preparations that may be used in the present invention can be found if U.S. Pat. No. 7,541,384 which is incorporated herein by reference.

The following embodiments are provided by way of example only and are by no means intended to be limiting.

### EXAMPLE 1

(R,R′) MNF was tested to evaluate its usefulness in attenuating inflammatory cytokines by the following method.

Cell Culture

Murine RAW 264.7 macrophages (ATCC TIB-71) were cultured in Dulbecco's Modified Eagle's Medium supplemented with fetal bovine serum (FBS) at a final concentration of 10%. The cells were maintained in T75 flasks in humidified atmosphere with 5% CO₂ at 37° C. Subculturing was performed every 2-3 days with a cellscraper.

Cell Treatment

RAW 264.7 macrophages were seeded onto 24-well plate in 1 mL of complete growth medium and left overnight to enable cell attachment. Next day the cells were washed with phosphate buffered saline and the medium was replaced with 0.5 mL of low serum medium containing 1% FBS. After 3 h of incubation, the medium was replaced with serum-free medium (0.5 mL) and incubated for 1.5 h. Then, the cells were exposed to 1 μM (R,R′)-4′-methoxy-1-naphthylfenoterol (MNF) or vehicle (DMSO) for 1 h followed by stimulation with 1 μg/mL lipopolysaccharide (LPS, E. coli 055: B5). Media were collected at the following timepoints after LPS administration: 0 h, 2 h, 4 h, 6 h, 8 h, and 10 h.

Assessment of TNF-α production

TNF-α was measured in all collected media samples using Quantikine ELISA kit (MTA00B) from R&D Systems according to manufacturer's protocol.

Statistical analysis Obtained data were assessed and plotted using GraphPad Prism 8.4.2. Two-way analysis of variance (ANOVA) followed by Sidak post-hoc test was used to compare mean TNF-α concentration across treatments and exposure times.

Results

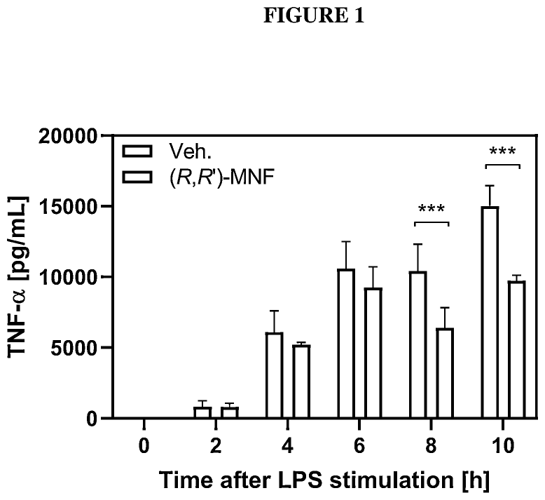

The results are shown in FIG. 1. The data in FIG. 1 demonstrated (R,R′) MNF elicited significant inhibition of LPS-induced production of TNF-α in RAW 264.7 macrophages at 8 and 10 h timepoint. This demonstrates a positive immunoinhibitory property of MNF.

The invention illustratively described herein suitably may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

What is claimed is:

1. A method for treating patients with viral infections comprising the step of administering a therapeutic amount of fenoterol and/or fenoterol analogues.

2. The method of claim 1 wherein the viral infection is a corona virus.

3. The method of claim 1 wherein the fenoterol and/or fenoterol analogue is 4′-methoxy-1-naphthylfenoterol, 4′-hydroxy-1-naphthylfenoterol, 4′-amino-1-naphthylfenoterol, 4′-methoxy-1-naphthylethylfenoterol, 4′-hydroxy-1-naphthylethylfenoterol, 4′-amino-1-naphthylethylfenoterol, 1-napthylfenoterol, 1-napthylethylfenoterol, 2-napthylfenoterol, 2-napthylethylfenoterol, pharmaceutically acceptable salts of the foregoing or isomers of the forgoing.

4. The method of claim 1 wherein the fenoterol and/or fenorerol analogue is administered orally, parenterally, transmucosally, optically, otically, nasally, via inhalation, transdermally or combination thereof

5. The method of claim 1 which further includes the administration of a second therapeutic agent selected from the group consisting of a high-mobility group box 1 (HMGB1) protein inhibitor, a Janus kinase (JAK) inhibitor, and immunosuppressant, an N-methyl-D-aspartate (NMDA) receptor inhibitor or combinations thereof.
